(19) United States
(12) Patent Application Publication
(10) Pub. No.: US 2024/0268711 A1
(43) Pub. Date: Aug. 15, 2024

(54) CONTACTLESS MONITORING OF RESPIRATORY RATE AND BREATHING ABSENCE USING FACE VIDEO

(71) Applicant: Samsung Electronics Co., Ltd., Suwon-si (KR)

(72) Inventors: Migyeong Gwak, Santa Clara, CA (US); Korosh Vatanparvar, San Jose, CA (US); Li Zhu, Saratoga, CA (US); Michael Chan, Atlanta, GA (US); Nafiul Rashid, San Jose, CA (US); Jungmok Bae, Menlo Park, CA (US); Jilong Kuang, San Jose, CA (US); Jun Gao, Menlo Park, CA (US)

(21) Appl. No.: 18/432,599

(22) Filed: Feb. 5, 2024

Related U.S. Application Data
(60) Provisional application No. 63/445,044, filed on Feb. 13, 2023.
(60) Provisional application No. 63/540,340, filed on Sep. 25, 2023.

(51) Int. Cl. A61B 5/113; A61B 5/00; A61B 5/08; G06V 40/16

(52) U.S. Cl. CPC A61B 5/113; A61B 5/08; A61B 5/7264; A61B 5/7275; G06V 40/171; G06V 2201/03

(57) ABSTRACT

A method includes capturing a video of a person's face using a camera. The method also includes determining a motion-based respiratory rate (RR) and a motion-based respiratory signal based on the video of the person's face. The method further includes determining a remote photoplethysmography (rPPG)-based RR and an rPPG-based respiratory signal based on the video of the person's face. The method also includes predicting whether the motion-based RR or the rPPG-based RR is more likely to be accurate using a trained machine learning model that receives the motion-based respiratory signal and the rPPG-based respiratory signal as input. In addition, the method includes presenting one of the motion-based RR or the rPPG-based RR based on the prediction.

### CROSS-REFERENCE TO RELATED APPLICATIONS AND PRIORITY CLAIM

This application claims priority under 35 U.S.C. § 119(e) to U.S. Provisional Patent Application No. 63/445,044 filed on Feb. 13, 2023 and U.S. Provisional Patent Application No. 63/540,340 filed on Sep. 25, 2023, both of which are hereby incorporated by reference in their entirety.

### TECHNICAL FIELD

This disclosure relates generally to machine learning systems and processes. More specifically, this disclosure relates to contactless monitoring of respiratory rate and breathing absence using face video.

### BACKGROUND

Respiratory rate (RR) is an important vital sign indicating overall respiratory system functionality and wellness. Among other things, respiratory rate is a reliable predictor of intensive care admission or death. It is also valuable information for patient care, especially for those with asthma, congestive heart failure, cardiac arrest, and breathlessness due to infection. Moreover, respiratory rate information can be useful in understanding fatigue, emotional status, or workout progress.

### SUMMARY

This disclosure relates to contactless monitoring of respiratory rate and breathing absence using face video.

In a first embodiment, a method includes capturing a video of a person's face using a camera. The method also includes determining a motion-based respiratory rate (RR) and a motion-based respiratory signal based on the video of the person's face. The method further includes determining a remote photoplethysmography (rPPG)-based RR and an rPPG-based respiratory signal based on the video of the person's face. The method also includes predicting whether the motion-based RR or the rPPG-based RR is more likely to be accurate using a trained machine learning model that receives the motion-based respiratory signal and the rPPG-based respiratory signal as input. In addition, the method includes presenting one of the motion-based RR or the rPPG-based RR based on the prediction.

In a second embodiment, an electronic device includes a camera configured to capture a video of a person's face. The electronic device also includes at least one processing device configured to determine a motion-based RR and a motion-based respiratory signal based on the video of the person's face. The at least one processing device is also configured to determine an rPPG-based RR and an rPPG-based respiratory signal based on the video of the person's face. The at least one processing device is further configured to predict whether the motion-based RR or the rPPG-based RR is more likely to be accurate using a trained machine learning model that receives the motion-based respiratory signal and the rPPG-based respiratory signal as input. In addition, the at least one processing device is configured to present one of the motion-based RR or the rPPG-based RR based on the prediction.

In a third embodiment, a non-transitory machine-readable medium contains instructions that when executed cause at least one processor of an electronic device to capture a video of a person's face using a camera. The non-transitory machine-readable medium also contains instructions that when executed cause the at least one processor to determine a motion-based RR and a motion-based respiratory signal based on the video of the person's face. The non-transitory machine-readable medium further contains instructions that when executed cause the at least one processor to determine an rPPG-based RR and an rPPG-based respiratory signal based on the video of the person's face. The non-transitory machine-readable medium also contains instructions that when executed cause the at least one processor to predict whether the motion-based RR or the rPPG-based RR is more likely to be accurate using a trained machine learning model that receives the motion-based respiratory signal and the rPPG-based respiratory signal as input. In addition, the non-transitory machine-readable medium contains instructions that when executed cause the at least one processor to present one of the motion-based RR or the rPPG-based RR based on the prediction.

Other technical features may be readily apparent to one skilled in the art from the following figures, descriptions, and claims.

Before undertaking the DETAILED DESCRIPTION below, it may be advantageous to set forth definitions of certain words and phrases used throughout this patent document. The terms “transmit,” “receive,” and “communicate,” as well as derivatives thereof, encompass both direct and indirect communication. The terms “include” and “comprise,” as well as derivatives thereof, mean inclusion without limitation. The term “or” is inclusive, meaning and/or. The phrase “associated with,” as well as derivatives thereof, means to include, be included within, interconnect with, contain, be contained within, connect to or with, couple to or with, be communicable with, cooperate with, interleave, juxtapose, be proximate to, be bound to or with, have, have a property of, have a relationship to or with, or the like.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

As used here, terms and phrases such as “have,” “may have,” “include,” or “may include” a feature (like a number, function, operation, or component such as a part) indicate the existence of the feature and do not exclude the existence of other features. Also, as used here, the phrases “A or B,” “at least one of A and/or B,” or “one or more of A and/or B” may include all possible combinations of A and B. For example, “A or B,” “at least one of A and B,” and “at least one of A or B” may indicate all of (1) including at least one A, (2) including at least one B, or (3) including at least one A and at least one B. Further, as used here, the terms “first” and “second” may modify various components regardless of importance and do not limit the components. These terms are only used to distinguish one component from another. For example, a first user device and a second user device may indicate different user devices from each other, regardless of the order or importance of the devices. A first component may be denoted a second component and vice versa without departing from the scope of this disclosure.

It will be understood that, when an element (such as a first element) is referred to as being (operatively or communicatively) “coupled with/to” or “connected with/to” another element (such as a second element), it can be coupled or connected with/to the other element directly or via a third element. In contrast, it will be understood that, when an element (such as a first element) is referred to as being “directly coupled with/to” or “directly connected with/to” another element (such as a second element), no other element (such as a third element) intervenes between the element and the other element.

As used here, the phrase “configured (or set) to” may be interchangeably used with the phrases “suitable for,” “having the capacity to,” “designed to,” “adapted to,” “made to,” or “capable of” depending on the circumstances. The phrase “configured (or set) to” does not essentially mean “specifically designed in hardware to.” Rather, the phrase “configured to” may mean that a device can perform an operation together with another device or parts. For example, the phrase “processor configured (or set) to perform A, B, and C” may mean a generic-purpose processor (such as a CPU or application processor) that may perform the operations by executing one or more software programs stored in a memory device or a dedicated processor (such as an embedded processor) for performing the operations.

The terms and phrases as used here are provided merely to describe some embodiments of this disclosure but not to limit the scope of other embodiments of this disclosure. It is to be understood that the singular forms “a,” “an,” and “the” include plural references unless the context clearly dictates otherwise. All terms and phrases, including technical and scientific terms and phrases, used here have the same meanings as commonly understood by one of ordinary skill in the art to which the embodiments of this disclosure belong. It will be further understood that terms and phrases, such as those defined in commonly-used dictionaries, should be interpreted as having a meaning that is consistent with their meaning in the context of the relevant art and will not be interpreted in an idealized or overly formal sense unless expressly so defined here. In some cases, the terms and phrases defined here may be interpreted to exclude embodiments of this disclosure.

Examples of an “electronic device” according to embodiments of this disclosure may include at least one of a smartphone, a tablet personal computer (PC), a mobile phone, a video phone, an e-book reader, a desktop PC, a laptop computer, a netbook computer, a workstation, a personal digital assistant (PDA), a portable multimedia player (PMP), an MP3 player, a mobile medical device, a camera, or a wearable device (such as smart glasses, a head-mounted device (HMD), electronic clothes, an electronic bracelet, an electronic necklace, an electronic accessory, an electronic tattoo, a smart mirror, or a smart watch). Other examples of an electronic device include a smart home appliance. Examples of the smart home appliance may include at least one of a television, a digital video disc (DVD) player, an audio player, a refrigerator, an air conditioner, a cleaner, an oven, a microwave oven, a washer, a dryer, an air cleaner, a set-top box, a home automation control panel, a security control panel, a TV box (such as SAMSUNG HOMESYNC, APPLETV, or GOOGLE TV), a smart speaker or speaker with an integrated digital assistant (such as SAMSUNG GALAXY HOME, APPLE HOMEPOD, or AMAZON ECHO), a gaming console (such as an XBOX, PLAYSTATION, or NINTENDO), an electronic dictionary, an electronic key, a camcorder, or an electronic picture frame. Still other examples of an electronic device include at least one of various medical devices (such as diverse portable medical measuring devices (like a blood sugar measuring device, a heartbeat measuring device, or a body temperature measuring device), a magnetic resource angiography (MRA) device, a magnetic resource imaging (MRI) device, a computed tomography (CT) device, an imaging device, or an ultrasonic device), a navigation device, a global positioning system (GPS) receiver, an event data recorder (EDR), a flight data recorder (FDR), an automotive infotainment device, a sailing electronic device (such as a sailing navigation device or a gyro compass), avionics, security devices, vehicular head units, industrial or home robots, automatic teller machines (ATMs), point of sales (POS) devices, or Internet of Things (IoT) devices (such as a bulb, various sensors, electric or gas meter, sprinkler, fire alarm, thermostat, street light, toaster, fitness equipment, hot water tank, heater, or boiler). Other examples of an electronic device include at least one part of a piece of furniture or building/structure, an electronic board, an electronic signature receiving device, a projector, or various measurement devices (such as devices for measuring water, electricity, gas, or electromagnetic waves). Note that, according to various embodiments of this disclosure, an electronic device may be one or a combination of the above-listed devices. According to some embodiments of this disclosure, the electronic device may be a flexible electronic device. The electronic device disclosed here is not limited to the above-listed devices and may include new electronic devices depending on the development of technology.

In the following description, electronic devices are described with reference to the accompanying drawings, according to various embodiments of this disclosure. As used here, the term “user” may denote a human or another device (such as an artificial intelligent electronic device) using the electronic device.

Definitions for other certain words and phrases may be provided throughout this patent document. Those of ordinary skill in the art should understand that in many if not most instances, such definitions apply to prior as well as future uses of such defined words and phrases.

None of the description in this application should be read as implying that any particular element, step, or function is an essential element that must be included in the claim scope. The scope of patented subject matter is defined only by the claims. Moreover, none of the claims is intended to invoke 35 U.S.C. § 112(f) unless the exact words “means for” are followed by a participle. Use of any other term, including without limitation “mechanism,” “module,” “device,” “unit,” “component,” “element,” “member,” “apparatus,” “machine,” “system,” “processor,” or “controller,” within a claim is understood by the Applicant to refer to structures known to those skilled in the relevant art and is not intended to invoke 35 U.S.C. § 112(f).

### BRIEF DESCRIPTION OF THE DRAWINGS

For a more complete understanding of this disclosure and its advantages, reference is now made to the following description taken in conjunction with the accompanying drawings, in which like reference numerals represent like parts:

FIG. 1 illustrates an example network configuration including an electronic device according to this disclosure;

FIG. 2 illustrates an example process for contactless monitoring of respiratory rate using face video according to this disclosure;

FIG. 3 illustrates an example video frame in which a face region has been identified according to this disclosure;

FIGS. 4A and 4B illustrate example charts showing feature extraction from motion-based and rPPG-based respiratory signals for machine learning-based respiratory rate selection according to this disclosure;

FIG. 5 illustrates an example process for detection of breathing absence using face video according to this disclosure; and

FIG. 6 illustrates an example method for contactless monitoring of respiratory rate and breathing absence using face video according to this disclosure.

### DETAILED DESCRIPTION

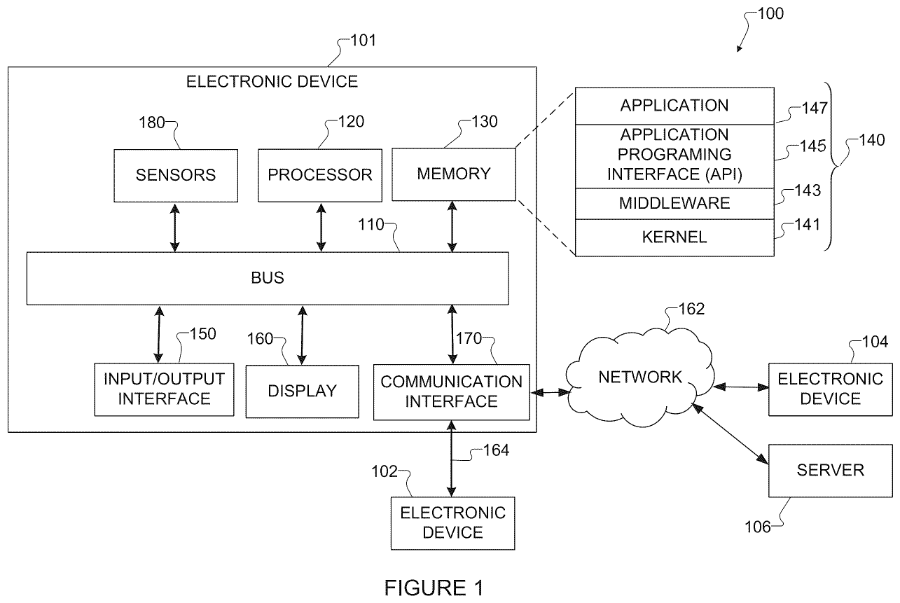

FIGS. 1 through 6, discussed below, and the various embodiments of this disclosure are described with reference to the accompanying drawings. However, it should be appreciated that this disclosure is not limited to these embodiments and all changes and/or equivalents or replacements thereto also belong to the scope of this disclosure.

As discussed above, respiratory rate (RR) is an important vital sign indicating overall respiratory system functionality and wellness. Among other things, respiratory rate is a reliable predictor of intensive care admission or death. It is also valuable information for patient care, especially for those with asthma, congestive heart failure, cardiac arrest, and breathlessness due to infection. Moreover, respiratory rate information can be useful in understanding fatigue, emotional status, or workout progress.

Many conventional RR monitoring devices require direct contact with human skin. Wearable sensors are expected to be directly attached to an individual's body, such as the face, torso, wrist, or finger. Available commercialized devices for respiration monitoring include a chest belt, smartwatch, face mask, pulse oximeter, nostril sensor, and wristband. A chest strap measures rib cage movements with a capacitive sensor. An optical sensor on a smartwatch or pulse oximeter can measure RR based on photoplethysmography (PPG). Recently, inertial measurement unit (IMU) sensors on earbuds have been used to measure RR. However, contact-based measurement is not appropriate for populations with sensitive skin, such as premature neonates and the elderly. It is also cumbersome for patients who need to wear on-body sensors for long-term monitoring. In addition, sharing contaminated sensors poses an extreme risk of spreading disease in hospitals and assisted living facilities.

A contactless RR measurement can be obtained using wireless signals, such as acoustic or radio-frequency signals. For example, a person's respiration state can be acquired by receiving a continuous propagated wave, which is influenced by repetitive chest movements while breathing. As a particular example, an ultra-wideband (UWB) radar-based system has been used to detect the respiration patterns of multiple persons. However, estimating RR using wireless signals often has limitations. For instance, the signal emitter should be located close to the human body, and the measurement is mainly optimized for indoor settings.

Camera-based respiratory monitoring is receiving growing interest as a non-contact approach and is being developed to take advantage of recent advanced cameras and image processing technologies. Infrared thermography, also known as thermal imaging, is one method of camera-based respiration monitoring. Infrared thermography captures radiation naturally emitted from the human skin. Some studies have extracted respiratory signs through thermal airflow variations at a person's nostrils using a far-infrared (FIR) camera. Moreover, depth cameras can be used to estimate breathing rates during sleep by recording chest movements. Both infrared and depth cameras do not need any light source, but they are high-end products and are excessively expensive. Consumer-accessible cameras are challenged by low pixel resolutions and low sampling rates, and they are typically unavailable on personal consumer-level devices.

Visually capturing respiratory-induced motions of a person's ribcage is another direct method of observing respiratory status. Various camera-based RR estimation approaches attempt to obtain motion signals of a person's chest region. However, a person's chest region is not always accessible in a facial video. Extracting a chest motion signal from a video is a challenge because there are no unique feature points in the chest region to be recognized when covered by various clothing items. As a result, identification of a person's chest often relies on face detection.

Besides RR estimation, detection of breathing absence is an important feature for monitoring breathing activity. Apnea is a suspension in breathing rhythm, and there are two types of sleep apnea. Obstructive sleep apnea occurs when the extrathoracic upper airway is blocked, and central sleep apnea occurs when the brain stem respiratory motor output is missing. The main difference between these two types of apneic events is that obstructive sleep apnea persists in the respiratory movement of the torso. In contrast, central sleep apnea does not involve any respiratory motion. The human head-neck system, which is biomechanically connected to the torso, is also influenced by respiratory motion. When respiratory-induced torso motion is reduced, unconstrained head motion as a sequence of the torso motion also decreases. Therefore, both types of apneas can be observed by the reduction or cessation of respiratory-induced head movement.

This disclosure provides various techniques for contactless monitoring of respiratory rate and breathing absence using face video. As described in more detail below, the disclosed embodiments can determine a motion-based RR based on a video of the person's face captured using a camera. The disclosed embodiments can also determine a remote photoplethysmography (rPPG)-based RR based on the video of the person's face. A pre-trained machine learning model can select between the motion-based RR or the rPPG-based RR to maintain accuracy with various measurement situations. Note that while some of the embodiments discussed below are described in the context of use in consumer electronic devices (such as smartphones), this is merely one example. It will be understood that the principles of this disclosure may be implemented in any number of other suitable contexts and may use any suitable devices.

FIG. 1 illustrates an example network configuration 100 including an electronic device according to this disclosure. The embodiment of the network configuration 100 shown in FIG. 1 is for illustration only. Other embodiments of the network configuration 100 could be used without departing from the scope of this disclosure.

According to embodiments of this disclosure, an electronic device 101 is included in the network configuration 100. The electronic device 101 can include at least one of a bus 110, a processor 120, a memory 130, an input/output (I/O) interface 150, a display 160, a communication interface 170, or a sensor 180. In some embodiments, the electronic device 101 may exclude at least one of these components or may add at least one other component. The bus 110 includes a circuit for connecting the components 120-180 with one another and for transferring communications (such as control messages and/or data) between the components.

The processor 120 includes one or more processing devices, such as one or more microprocessors, microcontrollers, digital signal processors (DSPs), application specific integrated circuits (ASICs), or field programmable gate arrays (FPGAs). In some embodiments, the processor 120 includes one or more of a central processing unit (CPU), an application processor (AP), a communication processor (CP), a graphics processor unit (GPU), or a neural processing unit (NPU). The processor 120 is able to perform control on at least one of the other components of the electronic device 101 and/or perform an operation or data processing relating to communication or other functions. As described in more detail below, the processor 120 may perform one or more operations for contactless monitoring of respiratory rate and breathing absence using face video.

The memory 130 can include a volatile and/or non-volatile memory. For example, the memory 130 can store commands or data related to at least one other component of the electronic device 101. According to embodiments of this disclosure, the memory 130 can store software and/or a program 140. The program 140 includes, for example, a kernel 141, middleware 143, an application programming interface (API) 145, and/or an application program (or “application”) 147. At least a portion of the kernel 141, middleware 143, or API 145 may be denoted an operating system (OS).

The kernel 141 can control or manage system resources (such as the bus 110, processor 120, or memory 130) used to perform operations or functions implemented in other programs (such as the middleware 143, API 145, or application 147). The kernel 141 provides an interface that allows the middleware 143, the API 145, or the application 147 to access the individual components of the electronic device 101 to control or manage the system resources. The application 147 may support one or more functions for contactless monitoring of respiratory rate and breathing absence using face video as discussed below. These functions can be performed by a single application or by multiple applications that each carry out one or more of these functions. The middleware 143 can function as a relay to allow the API 145 or the application 147 to communicate data with the kernel 141, for instance. A plurality of applications 147 can be provided. The middleware 143 is able to control work requests received from the applications 147, such as by allocating the priority of using the system resources of the electronic device 101 (like the bus 110, the processor 120, or the memory 130) to at least one of the plurality of applications 147. The API 145 is an interface allowing the application 147 to control functions provided from the kernel 141 or the middleware 143. For example, the API 145 includes at least one interface or function (such as a command) for filing control, window control, image processing, or text control.

The I/O interface 150 serves as an interface that can, for example, transfer commands or data input from a user or other external devices to other component(s) of the electronic device 101. The I/O interface 150 can also output commands or data received from other component(s) of the electronic device 101 to the user or the other external device.

The display 160 includes, for example, a liquid crystal display (LCD), a light emitting diode (LED) display, an organic light emitting diode (OLED) display, a quantum-dot light emitting diode (QLED) display, a microelectromechanical systems (MEMS) display, or an electronic paper display. The display 160 can also be a depth-aware display, such as a multi-focal display. The display 160 is able to display, for example, various contents (such as text, images, videos, icons, or symbols) to the user. The display 160 can include a touchscreen and may receive, for example, a touch, gesture, proximity, or hovering input using an electronic pen or a body portion of the user.

The communication interface 170, for example, is able to set up communication between the electronic device 101 and an external electronic device (such as a first electronic device 102, a second electronic device 104, or a server 106). For example, the communication interface 170 can be connected with a network 162 or 164 through wireless or wired communication to communicate with the external electronic device. The communication interface 170 can be a wired or wireless transceiver or any other component for transmitting and receiving signals.

The wireless communication is able to use at least one of, for example, WiFi, long term evolution (LTE), long term evolution-advanced (LTE-A), 5th generation wireless system (5G), millimeter-wave or 60 GHz wireless communication, Wireless USB, code division multiple access (CDMA), wideband code division multiple access (WCDMA), universal mobile telecommunication system (UMTS), wireless broadband (WiBro), or global system for mobile communication (GSM), as a communication protocol. The wired connection can include, for example, at least one of a universal serial bus (USB), high definition multimedia interface (HDMI), recommended standard 232 (RS-232), or plain old telephone service (POTS). The network 162 or 164 includes at least one communication network, such as a computer network (like a local area network (LAN) or wide area network (WAN)), Internet, or a telephone network.

The electronic device 101 further includes one or more sensors 180 that can meter a physical quantity or detect an activation state of the electronic device 101 and convert metered or detected information into an electrical signal. For example, one or more sensors 180 can include one or more cameras or other imaging sensors for capturing images of scenes. The sensor(s) 180 can also include one or more buttons for touch input, a gesture sensor, a gyroscope or gyro sensor, an air pressure sensor, a magnetic sensor or magnetometer, an acceleration sensor or accelerometer, a grip sensor, a proximity sensor, a color sensor (such as a red green blue (RGB) sensor), a bio-physical sensor, a temperature sensor, a humidity sensor, an illumination sensor, an ultraviolet (UV) sensor, an electromyography (EMG) sensor, an electroencephalogram (EEG) sensor, an electrocardiogram (ECG) sensor, an infrared (IR) sensor, an ultrasound sensor, an iris sensor, or a fingerprint sensor. The sensor(s) 180 can further include an inertial measurement unit, which can include one or more accelerometers, gyroscopes, and other components. In addition, the sensor(s) 180 can include a control circuit for controlling at least one of the sensors included here. Any of these sensor(s) 180 can be located within the electronic device 101.

In some embodiments, the electronic device 101 can be a wearable device or an electronic device-mountable wearable device (such as an HMD). For example, the electronic device 101 may represent an AR wearable device, such as a headset with a display panel or smart eyeglasses. In other embodiments, the first external electronic device 102 or the second external electronic device 104 can be a wearable device or an electronic device-mountable wearable device (such as an HMD). In those other embodiments, when the electronic device 101 is mounted in the electronic device 102 (such as the HMD), the electronic device 101 can communicate with the electronic device 102 through the communication interface 170. The electronic device 101 can be directly connected with the electronic device 102 to communicate with the electronic device 102 without involving a separate network.

The first and second external electronic devices 102 and 104 and the server 106 each can be a device of the same or a different type from the electronic device 101. According to certain embodiments of this disclosure, the server 106 includes a group of one or more servers. Also, according to certain embodiments of this disclosure, all or some of the operations executed on the electronic device 101 can be executed on another or multiple other electronic devices (such as the electronic devices 102 and 104 or server 106). Further, according to certain embodiments of this disclosure, when the electronic device 101 should perform some function or service automatically or at a request, the electronic device 101, instead of executing the function or service on its own or additionally, can request another device (such as electronic devices 102 and 104 or server 106) to perform at least some functions associated therewith. The other electronic device (such as electronic devices 102 and 104 or server 106) is able to execute the requested functions or additional functions and transfer a result of the execution to the electronic device 101. The electronic device 101 can provide a requested function or service by processing the received result as it is or additionally. To that end, a cloud computing, distributed computing, or client-server computing technique may be used, for example. While FIG. 1 shows that the electronic device 101 includes the communication interface 170 to communicate with the external electronic device 104 or server 106 via the network 162 or 164, the electronic device 101 may be independently operated without a separate communication function according to some embodiments of this disclosure.

The server 106 can include the same or similar components 110-180 as the electronic device 101 (or a suitable subset thereof). The server 106 can support to drive the electronic device 101 by performing at least one of operations (or functions) implemented on the electronic device 101. For example, the server 106 can include a processing module or processor that may support the processor 120 implemented in the electronic device 101. As described in more detail below, the server 106 may perform one or more operations to support techniques for contactless monitoring of respiratory rate and breathing absence using face video.

Although FIG. 1 illustrates one example of a network configuration 100 including an electronic device 101, various changes may be made to FIG. 1. For example, the network configuration 100 could include any number of each component in any suitable arrangement. In general, computing and communication systems come in a wide variety of configurations, and FIG. 1 does not limit the scope of this disclosure to any particular configuration. Also, while FIG. 1 illustrates one operational environment in which various features disclosed in this patent document can be used, these features could be used in any other suitable system.

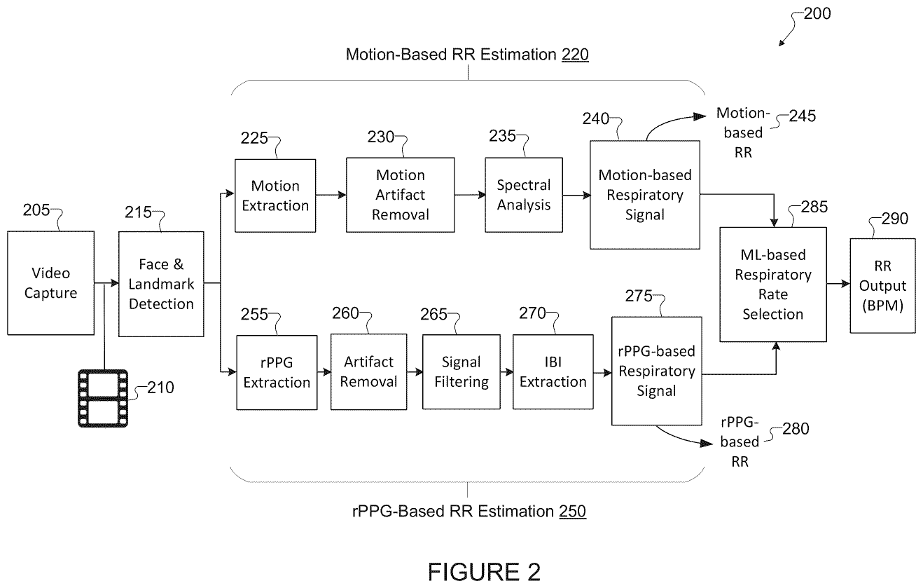

FIG. 2 illustrates an example process 200 for contactless monitoring of respiratory rate using face video according to this disclosure. For ease of explanation, the process 200 is described as being implemented using one or more components of the network configuration 100 of FIG. 1 described above, such as the electronic device 101. However, this is merely one example, and the process 200 could be implemented using any other suitable device(s) (such as the server 106) and in any other suitable system(s).

As shown in FIG. 2, the process 200 expands on two camera-based approaches that can be used to monitor respiratory information: rPPG-based RR measurement (which tracks skin color changes) and motion-based RR measurement (which tracks body movement). First, rPPG is proportional to the quantity of blood flowing through a person's blood vessels. This can be observed as subtle momentary changes in skin color as seen through an RGB camera or other camera. In some cases, the rPPG signal can be obtained by temporal RGB value changes of skin pixels in a video. The respiratory component can be extracted from pulsatile activity because the heart rate increases with inspiration and decreases with expiration, which is referred to as the respiratory sinus arrhythmia (RSA) relationship. It is noted that obtaining a clean rPPG signal can include overcoming motion artifacts, various illumination spectra, and different skin tones. Moreover, skin tissue typically needs to be visible to the camera to collect rPPG.

Second, motion-based RR can be measured by observing small repetitive movements of the respiratory system, like the lungs, the nose, the trachea, and the breathing muscles of a person. Because the RR is obtained by tracking the movement of selected pixels, detecting the skin tissue in a video may be unnecessary. Thus, the motion-based approach can estimate RR better than the rPPG-based approach when, for instance, a cap or a mask covers a person's face. It is noted that motion artifacts unrelated to respiratory-induced motion can negatively impact the measurement accuracy of motion-derived RR estimation. Furthermore, a lack of breathing motions can lead to an incorrect RR estimation.

The process 200 combines the rPPG-based and motion-based approaches to overcome the limitations of each modality and increase the overall performance. Thus, the process 200 provides a novel multimodal approach to monitoring respiratory activity using movement and color changes of the face as observed by a camera. As shown in FIG. 2, the process 200 includes a video capture operation 205 in which the electronic device 101 captures a video 210 of a person's face. The video capture operation 205 may be performed in response to an event, such as a user actuating a video capture control of the electronic device 101. The video capture operation 205 can be performed continuously, intermittently, repeatedly, on demand for a selected time period, or at any other suitable frequency and duration.

In some embodiments, the video 210 may be an RGB video captured using one imaging sensor 180 of the electronic device 101, such as a camera having an RGB sensor. In other embodiments, the video 210 may be captured using multiple imaging sensors 180 of the electronic device 101. Also, in some embodiments, the one or more imaging sensors 180 are positioned in front of the person's face at a distance of approximately 50 centimeters, although other distances and placements are possible. Further, in some embodiments, the frame rate of the video 210 is 30 or 60 frames per second (fps), although other frame rates are possible and within the scope of this disclosure.

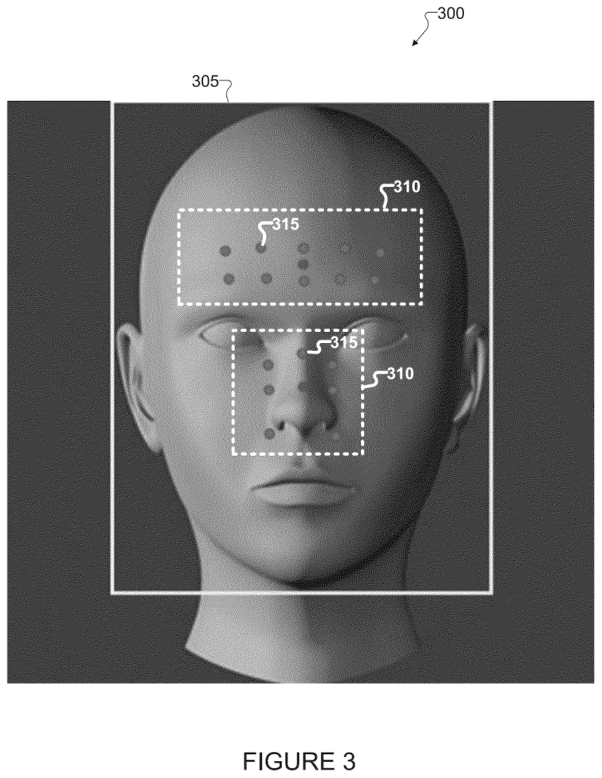

After capturing the video 210, the electronic device 101 performs a face and landmark detection operation 215. In the operation 215, the electronic device 101 searches frames of the video 210, such as by starting at an initial frame, for a rectangular or other region showing the person's face region. Any suitable technique may be used to detect the person's face, such as a deep-learning face detection algorithm or the Viola-Jones algorithm. If a face region is not found in the first frame, the electronic device 101 can move to successive frames until a frame with a face region is found. FIG. 3 illustrates an example video frame 300 in which a face region 305 has been identified according to this disclosure. Any background beside the face region 305 can be removed for further processing and to preserve privacy.

Once the face region 305 is identified, the electronic device 101 selects multiple facial landmarks 315 within the face region 305. In some embodiments, the electronic device 101 selects ten facial landmarks 315 in the person's forehead region and seven facial landmarks 315 in the person's nose region, although other numbers of landmarks may be used in each region. Also, in some embodiments, the facial landmarks 315 can be selected from a database of predetermined facial landmarks, although facial landmarks may be identified in any other suitable manner.

The electronic device 101 also selects multiple regions of interest (ROIs) within the face region 305 based on the selected landmarks 315. In some embodiments, the electronic device 101 selects two rectangular or other ROIs, namely (i) a first ROI 310 corresponding to the person's nose region and (ii) a second ROI 310 corresponding to the person's forehead region. The electronic device 101 can also select additional ROIs 310 for use in rPPG-based RR estimation. For example, the electronic device 101 can employ a Gaussian mixture model to identify skin pixels on the detected face region 305 and select multiple (such as 32) ROIs 310 using a skin likelihood score.

Once the electronic device 101 has detected the face region 305, the ROIs 310, and the facial landmarks 315 in the video 210, the electronic device 101 performs two separate RR estimation techniques, namely (i) motion-based RR estimation 220, and (ii) rPPG-based RR estimation 250. Motion-based RR estimation 220 includes a motion extraction operation 225 in which the electronic device 101 extracts a face motion signal by tracking the facial landmarks 315 over time. In some embodiments, the electronic device 101 uses a motion tracking algorithm to track the horizontal (X-axis) and vertical (Y-axis) movements of the facial landmarks 315 by detecting the X and Y coordinates of the center point of each facial landmark 315 in each frame of the video 210. In particular embodiments, the electronic device 101 only utilizes location changes in the Y-axis because a person's breathing motion is highly correlated with the vertical head movement during an upright posture. Any suitable technique can be used for motion tracking, such as the Lucas-Kanade-Tomasi (LKT) optical flow algorithm. The electronic device 101 can also use an overlapping sliding window approach to estimate RR every second. Accordingly, the motion signal can be buffered into a sliding window with a specified length (such as forty seconds) and a step size of one second.

In general, face motion signals can be vulnerable to noise or motion artifacts due to sudden voluntary or involuntary movements of the person during recording of the video 210. Thus, after the motion extraction operation 225, the electronic device 101 performs a motion artifact removal operation 230 to remove the motion artifacts from the motion signal. In the motion artifact removal operation 230, the electronic device 101 smooths the motion signal, such as with a moving average. The electronic device 101 also determines a motion speed signal by calculating the differences between successive values in the motion signal. Finally, the electronic device 101 uses the absolute values of the motion speed signal to define a threshold for motion artifact removal. Sudden motion artifacts have a higher speed than respiratory-induced motion of the head and chest. Therefore, the artifacts appear as outliers in the distribution of the motion speed signal. The electronic device 101 can utilize kurtosis or other technique to determine if a motion signal within a thirty-second or other window has sudden motion artifacts. A kurtosis-based motion artifact removal sets the noisy portion to zero based on a dynamic threshold. If the kurtosis is increased, the probability distribution has a thin “bell” shape, which is more concentrated about the mean. Therefore, the motion signal has more outliers when the kurtosis is larger than a selected value (such as three).

After the electronic device 101 identifies the existence of motion artifacts, the electronic device 101 can determine the outliers, such as based on a static or dynamic threshold. In some embodiments, a value of 0.35 can be selected as a static threshold based on observation of the distribution of magnitude signal values. Of course, other values are possible and within the scope of this disclosure. The top ten percent or other portion of the distribution of the absolute speed signal in the Y-axis can become the dynamic threshold in each window. In some cases, only the speed signal in the Y-axis may be used since respiration mostly affects the vertical movement of the face or chest. Any motions in the X-axis are more likely to be noise during a voluntary motion. Therefore, a Y-axis speed value beyond the threshold may be considered an outlier and can be replaced with zero, which is analogous to replacing sudden movements with a breath holding.

After removal of the motion artifacts, the electronic device 101 uses spectral analysis 235 to determine a motion-based respiratory signal 240 and estimate an instantaneous motion-based RR 245. For example, the electronic device 101 may remove the linear trend of the cleaned speed signal and use a moving average technique to make the signal smooth. In some embodiments, a second-order Savitzky-Golay filter with a two-second subset window or other window can be applied for further smoothing of the signal. The electronic device 101 may use a filter (such as a Butterworth filter with cut-off frequencies of f_{c1}=0.05 Hz and f_{c2}=0.75 Hz using a Hamming window) to extract a signal within a frequency spectrum related to breathing. The filtered signal corresponds to the motion-based respiratory signal 240 in a forty-second window or other window. The motion-based respiratory signal 240 can be normalized, such as by using the Frobenius norm, and converted, such as by using a discrete Fourier Transform (DFT) with zero padding. The electronic device 101 can estimate RR, such as from 3 to 45 breaths per minute (BPM), from the frequency domain signal to avoid excessively incorrect estimation. By observing the DFT signal, the frequency component with the highest peak may correspond to the instantaneous RR. Instant RR can be measured for all landmarks accordingly. A signal-to-noise ratio (SNR) can determine the highly-correlated signal waveform to respiration. The electronic device 101 can therefore select the RR with the highest SNR among the measured RRs from multiple landmarks as the motion-based RR 245.

In rPPG-based RR estimation 250, the electronic device 101 performs an rPPG extraction operation 255 in which rPPG signals are extracted from the ROIs 310 of the video 210. Any suitable technique can be used to extract the rPPG signals. In some embodiments, the electronic device 101 can use the chrominance (CHROM) method to extract rPPG signals from each ROI 310.

After extracting the rPPG signals from each ROI 310, the electronic device 101 performs an artifact removal operation 260. In some ROIs 310, camera artifacts (such as those that are produced by a smartphone camera) can be stronger than the cardiac pulsation of the videoed person. In other ROIs 310, the camera artifacts are weaker and barely noticeable. To remove the camera artifacts, the electronic device 101 may check an rPPG signal from an ROI 310 for the existence of strong harmonics. If the power spectral density (PSD) of the second harmonic (such as at 2 Hz) is higher than the dominant PSD (such as at 1 Hz) multiplied by a coefficient, the rPPG signal may be classified as one that contains strong camera artifacts and could be discarded. After artifact removal, the rPPG signals from multiple ROIs can be combined into a weighted rPPG signal, such as by using an SNR-based weighted average.

The electronic device 101 also performs a signal filtering operation 265. In some embodiments, the electronic device 101 applies a filter (such as a comb notch filter) to further suppress the weighted rPPG signal with a fundamental frequency of 1 Hz if the cardiac activity is not pulsating around 1 Hz. The electronic device 101 may also apply a narrower filter with a bandwidth using coarse heart rate and respiratory rate (such as a “HR-RR-tuned filter”) on the weighted rPPG signal.

The electronic device 101 performs IBI extraction 270 using the weighted rPPG signal to generate an inter-beat interval (IBI) signal. IBI is defined as the distance between consecutive heartbeats in rPPG, such as in milliseconds. One of the main fluctuations in heart rate is caused by respiratory sinus arrhythmia (RSA). IBI values decrease with inspiration and increase with expiration. The IBI signal is considered to be a respiratory signal that can be used to calculate an rPPG-based RR 280. In some embodiments, the electronic device 101 can use peak detection to generate the IBI signal.

Because the IBI signal provides a more explicit RSA relationship than a filtered rPPG signal, the electronic device 101 selects interpolated IBI signals as an rPPG-based respiratory signal 275 and estimates the rPPG-based RR 280. A linear trend in the IBI signal may be removed to reduce low-frequency noise. In some embodiments, the electronic device 101 can employ a linear interpolation so that the rPPG-based respiratory signal 275 has the same sample size as the motion-based respiratory signal 240. The electronic device 101 can normalize the rPPG-based respiratory signal 275, such as by using the Frobenius norm, and convert the rPPG-based respiratory signal 275, such as by using a DFT with zero padding. The electronic device 101 can estimate the rPPG-based RR 280, such as from 3 to 45 BPM, from the frequency domain signal to avoid excessively incorrect estimation.

The results of the motion-based RR estimation 220 and the rPPG-based estimation RR 250 include two independent respiratory signals (the motion-based respiratory signal 240 and the rPPG-based respiratory signal 275) and two RR values (the motion-based RR 245 and the rPPG-based RR 280). The electronic device 101 can perform a respiratory rate selection operation 285 to predict whether the motion-based RR 245 or the rPPG-based RR 280 is more likely to be accurate and can select the more accurate rate, which the electronic device 101 can output, display, or otherwise present as a RR output 290. In some embodiments, the electronic device 101 uses a trained machine learning model, such as a lightweight machine learning classifier, to select between the motion-based RR 245 and the rPPG-based RR 280. For example, if the absolute difference between the two RR values (the motion-based RR 245 and the rPPG-based RR 280) is larger than a specified value (such as 2 BPM) and the sample size of the IBI signal is larger than another specified value (such as 19), the electronic device 101 may apply the trained ML model. Otherwise, the signal quality of the rPPG may be considered to be insufficient, and the electronic device 101 may select the motion-based RR 245 as a default selection. For post-processing of continuous RR estimation, a seven-point median smoothing or other smoothing operation can be employed to reduce random noise before finalizing the RR.

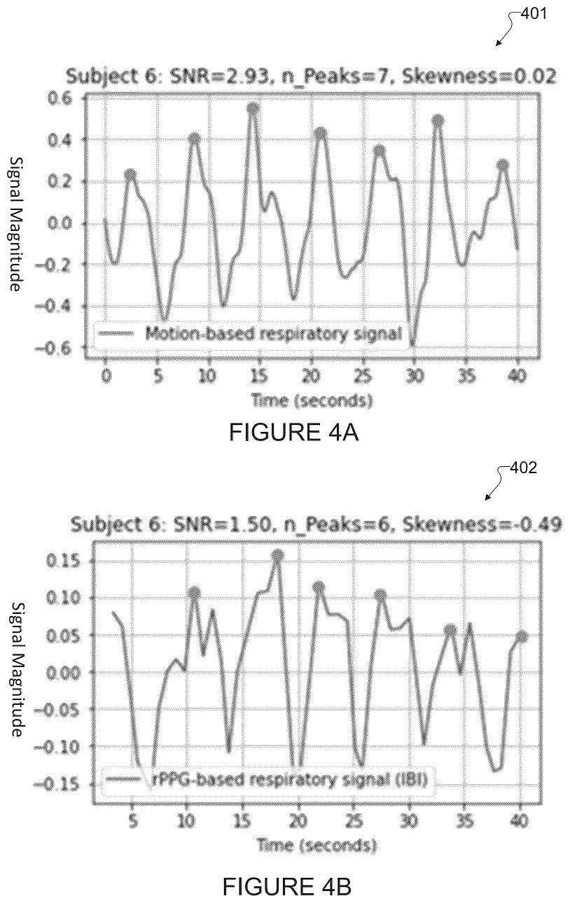

As input to the ML model, the electronic device 101 may extract multiple features from each windowed respiratory signal 240 and 275, such as SNR, number of peaks, and skewness. These features can represent the signal quality of the respiratory signals 240 and 275. For example, FIGS. 4A and 4B illustrate example charts 401 and 402 showing feature extraction from motion-based and rPPG-based respiratory signals for machine learning-based RR selection according to this disclosure. In particular, a chart 401 in FIG. 4A depicts an example motion-based respiratory signal 240 over a forty second time window, and a chart 402 in FIG. 4B depicts an example rPPG-based respiratory signal 275 over the forty second window.

As shown in FIGS. 4A and 4B, SNR, number of peaks, and skewness can be identified from the signals 240 and 275. SNR determines the highly correlated signal waveform to respiration and may be calculated from the PSD of each respiratory signal 240 and 275. The number of peaks on a periodic respiratory signal can be directly associated with RR. In some embodiments, the electronic device 101 may apply the same peak detection algorithm that is used for IBI detection. Skewness is a measurement of the asymmetry of the probability distribution. The skewness index has been outperformed among eight signal quality indices (SQI) for PPG signals. The shape of a single waveform of the respiratory signals 240 and 275 is unlike the PPG signals, but the skewness index can determine if there is distortion on the window signal. The skewness index can increase when the window signal has a weak or irregular waveform. The number of peaks and skewness can be calculated in the time domain.

As discussed above, in some embodiments, the ML model can be a binary classification model. The classification model can be trained to determine the final output between two calculated RRs. To train the ML model, the electronic device 101 (or the server 106 or other device) can access a dataset that includes multiple training samples. In some embodiments, each training sample includes a motion-based respiratory signal, an rPPG-based respiratory signal, and a label indicating whether a motion-based RR or an rPPG-based RR is closer to a ground truth RR for that training sample. Also, in some embodiments, the label for each training sample is the modality name with the smaller error on the calculated RR. Further, in some embodiments, the electronic device 101, server 106, or other device can divide the dataset into a training set and a testing set, such as with a ratio of 2:1. Thus, only a subset of the entire dataset may be used for training to avoid overfitting.

For each of the training samples in the training set, the electronic device 101, server 106, or other device performs the training. In particular, the electronic device 101, server 106, or other device extracts features of the motion-based respiratory signal and the rPPG-based respiratory signal and provides the features as input to the ML model, which predicts whether the motion-based RR or the rPPG-based RR is more likely to be closer to the ground truth RR. The ML classifier can be trained using any suitable set of features. In some embodiments, the features can include SNR, number of peaks, and skewness. The electronic device 101, server 106, or other device updates one or more parameters or weights of the ML model based on a comparison of the label and the prediction. In some cases, a class weight of 9 to 1 for rPPG-derived RR and motion-derived RR can be applied to the decision tree to resolve any class imbalance issues in the feature set.

Although FIGS. 2 through 4B illustrate one example of a process 200 for contactless monitoring of respiratory rate using face video and related details, various changes may be made to FIGS. 2 through 4B. For example, while the process 200 is described as involving specific sequences of operations, various operations described with respect to FIG. 2 could overlap, occur in parallel, occur in a different order, or occur any number of times (including zero times). Also, the specific operations shown in FIG. 2 are examples only, and other techniques could be used to perform each of the operations shown in FIG. 2. As a particular example, instead of kurtosis, skewness can be used to determine signal distortion because skewness measures the asymmetry of the distributed values in the windowed signal. Thus, for instance, the top ten percent of the distribution of the absolute speed signal in the Y-axis can be used as the dynamic threshold in each window. A Y-axis speed value beyond the threshold may be considered an outlier and may be replaced with zero, which may be similar to replacing any sudden motions to breath-holding. Also, instead of using a second-order Savitzky-Golay filter, a Butterworth filter can be used for smoothing the motion signal.

It should be noted that estimation of RR with spectral analysis, such as is described with respect to FIG. 2, may not detect an event of breath holding because peaks of the power spectrum cannot be zero if any motion noise exists in the signal. Therefore, an ML-based breathing absence detector algorithm may be used to identify an apnea event and improve overall RR estimation accuracy.

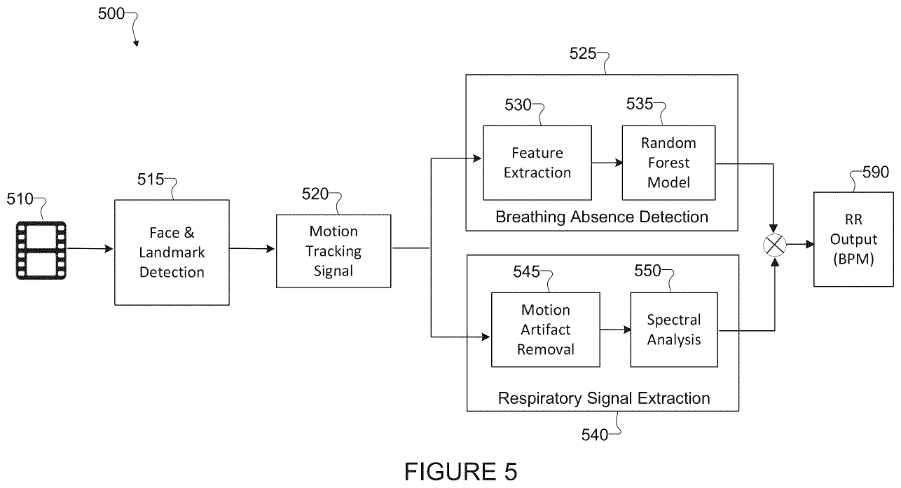

FIG. 5 illustrates an example process 500 for detection of breathing absence using face video according to this disclosure. For case of explanation, the process 500 is described as being implemented using one or more components of the network configuration 100 of FIG. 1 described above, such as the electronic device 101. However, this is merely one example, and the process 500 could be implemented using any other suitable device(s) (such as the server 106) and in any other suitable system(s).

As shown in FIG. 5, the process 500 includes multiple components that may be the same as or similar to corresponding components of the process 200 of FIG. 2. In some embodiments, the process 500 and the process 200 can be performed together, in sequence or in parallel, in order to provide a more robust respiration evaluation solution. In the process 500, the electronic device 101 captures a video 510 showing a person's face.

The electronic device 101 performs a face and landmark detection operation 515 on the video 510 to detect the person's face region, multiple ROIs, and multiple facial landmarks. The operation 515 can be the same as or similar to the face and landmark detection operation 215 of FIG. 2. In some embodiments, the electronic device 101 can implement an ML model to detect the face region and facial landmarks. Each frame of the video 510 can be analyzed using a face detection algorithm. When a face region is detected, the background can be removed to reduce image processing costs and possibly incorrect face detection. The average location of a set of landmarks in the forehead region and a set of landmarks in the nose region may be determined in each frame.

The electronic device 101 tracks the facial landmarks over time to generate a motion tracking signal 520 representing head movement. A robust motion tracking signal 520 is useful for obtaining respiratory-related information from the video 510. In some embodiments, the electronic device 101 can determine the location changes of the landmarks in the X-Y coordinates, frame by frame, to generate the motion tracking signal 520. If needed, the face and landmark detection operation 515 may be performed again if the detected face moves out of the frame.

The electronic device 101 also performs breathing absence detection 525 using a sliding window of the motion tracking signal 520. In some embodiments, the electronic device 101 may use a seven-second sliding window approach with a one-second interval. Note, however, that other window sizes (such as six or eight seconds) and other intervals (such as two or three seconds) may be possible. The breathing absence detection 525 includes a feature extraction operation 530. In the feature extraction operation 530, the electronic device 101 generates multiple signals, such as a normalized signal, a filtered signal, and a speed signal, from the motion tracking signal 520. The raw motion tracking signal 520 of each window can be normalized by removing the linear trend of the signal, resulting in the normalized signal. The electronic device 101 may use a filter (such as a second-order Butterworth filter with 0.05 and 0.75 cut-off frequencies) to create the filtered signal. The speed signal may represent the difference between successive values of the smoothed normalized signal by a moving average.

The electronic device 101 extracts statistical features from the normalized signal, the filtered signal, and the speed signal in the time domain. Statistical features represent characteristics of the signals, such as mean, variance, standard deviation, minimum, maximum, absolute maximum, averaged second power, range, median, root mean square, crest factor, skewness, kurtosis, or any combination thereof. The electronic device 101 also extends the normalized signal with zero padding and transforms the normalized signal, such as with a fast Fourier transform (FFT), to get features in the frequency domain. The electronic device 101 can calculate the same statistical features from the power spectrum, such as with a frequency range between 3 and 45 BPM.

Once the electronic device 101 obtains the various features, the electronic device 101 feeds the extracted features into a random forest classifier model 535 that is trained for breathing absence detection. In some embodiments, the random forest classifier model 535 uses averaging of multiple decision tree classifiers that have been trained on various sub-samples of a training dataset. In some embodiments, an apneic event is defined as a suspension in breathing activity for more than a predetermined duration (such as 9 seconds, 10 seconds, 11 seconds, or other duration). Consecutive breath-holding classification results may be aggregated to detect an apnea episode.

The electronic device 101 also performs respiratory signal extraction 540 using a sliding window of the motion tracking signal 520. The respiratory signal extraction 540 can include a motion artifact removal operation 545 (which can be the same as or similar to the motion artifact removal operation 230) and spectral analysis 550 (which can be the same as or similar to the spectral analysis 235). The motion artifact removal operation 545 may be used to determine if the motion tracking signal 520 has any voluntary head movement. When the kurtosis of the speed signal is bigger than a specified value (such as three), the window signal may be excluded. A RR can be calculated using the results of the spectral analysis 550. A final RR output 590 can be determined by combining the RR with the results from the breathing absence detection 525.

The random forest classifier model 535 can be trained using a dataset of training videos. In some embodiments, the dataset can be collected while subjects are video-recorded while performing various tasks. These tasks may include breath-holding in which the subject holds his or her breath for a period of time (such as up to one minute) and has natural breathing for another period of time (such as ten seconds). These tasks may also include controlled breathing in which the subject watches a guided breathing video to perform controlled breathing at target rates (such as 5, 10, 15, 20, and 25 breaths per minute). These tasks may further include spontaneous breathing in lower light levels so that facial videos of spontaneous breathing are recorded at low illumination levels. The videos may be captured using a commercially-available RGB camera (such as the camera of a smartphone) or other imaging device(s). In some embodiments, to avoid any overfitting issues, the dataset can be separated into a training set and a testing set, such as with a ratio of 2:1.

Although FIG. 5 illustrates one example of a process 500 for detection of breathing absence using face video, various changes may be made to FIG. 5. For example, while the process 500 is described as involving specific sequences of operations, various operations described with respect to FIG. 5 could overlap, occur in parallel, occur in a different order, or occur any number of times (including zero times). Also, the specific operations shown in FIG. 5 are examples only, and other techniques could be used to perform each of the operations shown in FIG. 5.

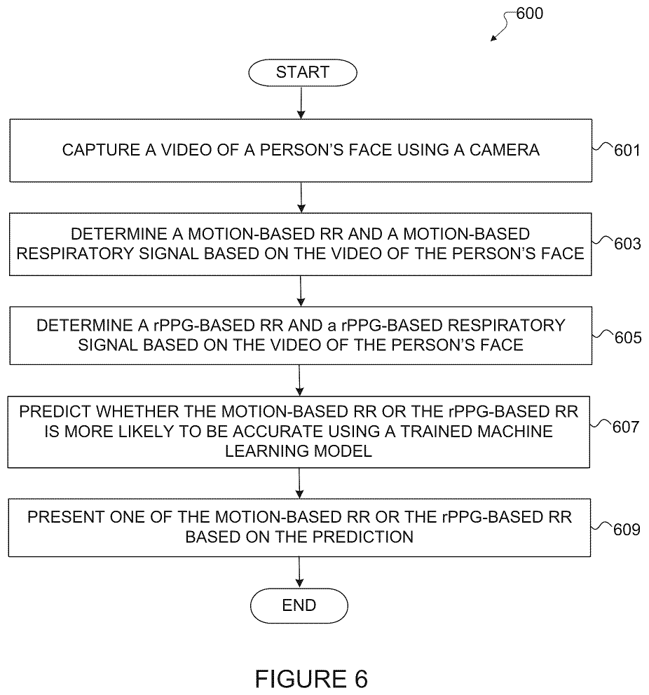

FIG. 6 illustrates an example method 600 for contactless monitoring of respiratory rate and breathing absence using face video according to this disclosure. For case of explanation, the method 600 shown in FIG. 6 is described as being performed using the electronic device 101 shown in FIG. 1 and the process 200 shown in FIG. 2. However, the method 600 shown in FIG. 6 could be used with any other suitable device(s) or system(s) and could be used to perform any other suitable process(es), such as the process 500 shown in FIG. 5.

As shown in FIG. 6, at step 601, a video of a person's face is captured using a camera. This could include, for example, the electronic device 101 capturing a video 210 of a person's face, such as is shown in FIG. 2. At step 603, a motion-based RR and a motion-based respiratory signal are determined based on the video of the person's face. This could include, for example, the electronic device 101 performing the motion-based RR estimation 220 to determine the motion-based respiratory signal 240 and the motion-based RR 245, such as is shown in FIG. 2.

At step 605, an rPPG-based RR and an rPPG-based respiratory signal are determined based on the video of the person's face. This could include, for example, the electronic device 101 performing the rPPG-based RR estimation 250 to determine the rPPG-based RR 280 and the rPPG-based respiratory signal 275, such as is shown in FIG. 2. At step 607, a trained ML model is used to predict whether the motion-based RR or the rPPG-based RR is more likely to be accurate. The ML model receives the motion-based respiratory signal and the rPPG-based respiratory signal as input. This could include, for example, the electronic device 101 performing the ML-based respiratory rate selection operation 285, such as is shown in FIG. 2. At step 609, the motion-based RR or the rPPG-based RR is presented based on the prediction. This could include, for example, the electronic device 101 displaying, transmitting, or otherwise outputting the RR output 290, such as is shown in FIG. 2.

Although FIG. 6 illustrates one example of a method 600 for contactless monitoring of respiratory rate and breathing absence using face video, various changes may be made to FIG. 6. For example, while shown as a series of steps, various steps in FIG. 6 could overlap, occur in parallel, occur in a different order, or occur any number of times (including zero times).

The disclosed embodiments are suitable for a wide variety of use cases. For instance, the disclosed embodiments enable any suitable consumer electronic device (such as a person's smartphone, smart television, tablet computer, or the like) to monitor a person's vital signs in real time. The vital signs can be monitored in a contactless manner since the user does not have to wear any sensors. The vital signs can be monitored during home exercise, during a video call (such as a call with a healthcare provider), or while sleeping. As a particular example, vital signs of a baby can be monitored as a part of neonatal or baby monitoring application.

Note that the operations and functions shown in or described with respect to FIGS. 2 through 6 can be implemented in an electronic device 101, 102, 104, server 106, or other device(s) in any suitable manner. For example, in some embodiments, the operations and functions shown in or described with respect to FIGS. 2 through 6 can be implemented or supported using one or more software applications or other software instructions that are executed by the processor 120 of the electronic device 101, 102, 104, server 106, or other device(s). In other embodiments, at least some of the operations and functions shown in or described with respect to FIGS. 2 through 6 can be implemented or supported using dedicated hardware components. In general, the operations and functions shown in or described with respect to FIGS. 2 through 6 can be performed using any suitable hardware or any suitable combination of hardware and software/firmware instructions.

Although this disclosure has been described with reference to various example embodiments, various changes and modifications may be suggested to one skilled in the art. It is intended that this disclosure encompass such changes and modifications as fall within the scope of the appended claims.

What is claimed is:

1. A method comprising:
capturing a video of a person's face using a camera;
determining a motion-based respiratory rate (RR) and a motion-based respiratory signal based on the video of the person's face;
determining a remote photoplethysmography (rPPG)-based RR and an rPPG-based respiratory signal based on the video of the person's face;
predicting whether the motion-based RR or the rPPG-based RR is more likely to be accurate using a trained machine learning model that receives the motion-based respiratory signal and the rPPG-based respiratory signal as input; and
presenting one of the motion-based RR or the rPPG-based RR based on the prediction.

2. The method of claim 1, wherein determining the motion-based RR and the motion-based respiratory signal based on the video of the person's face comprises:
identifying face landmarks on the person's face based on the video, wherein the face landmarks are on the person's forehead and the person's nose;
generating a motion signal based on vertical location changes of the face landmarks in the video; and
extracting the motion-based respiratory signal based on the motion signal using spectral analysis.

3. The method of claim 2, wherein determining the motion-based RR and the motion-based respiratory signal based on the video of the person's face further comprises:
removing artifacts from the motion signal using a kurtosis-based motion artifacts detection technique; and
smoothing the motion signal using a filter.

4. The method of claim 1, wherein determining the rPPG-based RR and the rPPG-based respiratory signal based on the video of the person's face comprises:
identifying regions of interest on the person's face;
extracting an rPPG signal for each region of interest based on the video;
extracting an inter-beat interval (IBI) signal based on a weighted combination of the rPPG signals corresponding to the regions of interest; and
extracting the rPPG-based respiratory signal based on the IBI signal.

5. The method of claim 1, wherein the machine learning model is a binary classifier model trained by:
accessing a training dataset comprising multiple training samples, each training sample including a motion-based respiratory signal, an rPPG-based respiratory signal, and a label indicating whether a motion-based RR or an rPPG-based RR is closer to a ground truth RR for that training sample; and
for each training sample:
extracting features of the motion-based respiratory signal and the rPPG-based respiratory signal;
providing the features as input to the machine learning model which predicts whether the motion-based RR or the rPPG-based RR is more likely to be closer to the ground truth RR; and
updating parameters of the machine learning model based on a comparison of the label and the prediction.

6. The method of claim 5, wherein the features include one or more of: a signal noise ratio, a number of peaks, and a skewness.

7. The method of claim 1, wherein the camera is coupled to a mobile device, a computer, or a television.

8. An electronic device comprising:
a camera configured to capture a video of a person's face; and
at least one processing device configured to:
determine a motion-based respiratory rate (RR) and a motion-based respiratory signal based on the video of the person's face;
determine a remote photoplethysmography (rPPG)-based RR and an rPPG-based respiratory signal based on the video of the person's face;
predict whether the motion-based RR or the rPPG-based RR is more likely to be accurate using a trained machine learning model that receives the motion-based respiratory signal and the rPPG-based respiratory signal as input; and
present one of the motion-based RR or the rPPG-based RR based on the prediction.

9. The electronic device of claim 8, wherein, to determine the motion-based RR and the motion-based respiratory signal based on the video of the person's face, the at least one processing device is configured to:
identify face landmarks on the person's face based on the video, wherein the face landmarks are on the person's forehead and the person's nose;
generate a motion signal based on vertical location changes of the face landmarks in the video; and
extract the motion-based respiratory signal using spectral analysis based on the motion signal.

10. The electronic device of claim 9, wherein, to determine the motion-based RR and the motion-based respiratory signal based on the video of the person's face, the at least one processing device is further configured to:
remove artifacts from the motion signal using a kurtosis-based motion artifacts detection technique; and
smooth the motion signal using a filter.

11. The electronic device of claim 8, wherein, to determine the rPPG-based RR and the rPPG-based respiratory signal based on the video of the person's face, the at least one processing device is configured to:
identify regions of interest on the person's face;
extract an rPPG signal for each region of interest based on the video;
extract an inter-beat interval (IBI) signal based on a weighted combination of the rPPG signals corresponding to the regions of interest; and
extract the rPPG-based respiratory signal based on the IBI signal.

12. The electronic device of claim 8, wherein:
the machine learning model is a binary classifier model; and
to train the machine learning model, the at least one processing device is configured to:
access a training dataset comprising multiple training samples, each training sample including a motion-based respiratory signal, an rPPG-based respiratory signal, and a label indicating whether a motion-based RR or an rPPG-based RR is closer to a ground truth RR for that training sample; and
for each training sample:
extract features of the motion-based respiratory signal and the rPPG-based respiratory signal;
provide the features as input to the machine learning model which predicts whether the motion-based RR or the rPPG-based RR is more likely to be closer to the ground truth RR; and
update parameters of the machine learning model based on a comparison of the label and the prediction.

13. The electronic device of claim 12, wherein the features include one or more of: a signal noise ratio, a number of peaks, and a skewness.

14. The electronic device of claim 8, wherein the electronic device comprises a mobile device, a computer, or a television.

15. A non-transitory machine-readable medium containing instructions that when executed cause at least one processor of an electronic device to:
capture a video of a person's face using a camera;
determine a motion-based respiratory rate (RR) and a motion-based respiratory signal based on the video of the person's face;
determine a remote photoplethysmography (rPPG)-based RR and an rPPG-based respiratory signal based on the video of the person's face;
predict whether the motion-based RR or the rPPG-based RR is more likely to be accurate using a trained machine learning model that receives the motion-based respiratory signal and the rPPG-based respiratory signal as input; and
present one of the motion-based RR or the rPPG-based RR based on the prediction.

16. The non-transitory machine-readable medium of claim 15, wherein the instructions that when executed cause the at least one processor to determine the motion-based RR and the motion-based respiratory signal based on the video of the person's face comprise:
instructions that when executed cause the at least one processor to:
identify face landmarks on the person's face based on the video, wherein the face landmarks are on the person's forehead and the person's nose;
generate a motion signal based on vertical location changes of the face landmarks in the video; and
extract the motion-based respiratory signal using spectral analysis based on the motion signal.

17. The non-transitory machine-readable medium of claim 16, wherein the instructions that when executed cause the at least one processor to determine the motion-based RR and the motion-based respiratory signal based on the video of the person's face further comprise:
instructions that when executed cause the at least one processor to:
remove artifacts from the motion signal using a kurtosis-based motion artifacts detection technique; and
smooth the motion signal using a filter.

18. The non-transitory machine-readable medium of claim 15, wherein the instructions that when executed cause the at least one processor to determine the rPPG-based RR and the rPPG-based respiratory signal based on the video of the person's face comprise:
instructions that when executed cause the at least one processor to:
identify regions of interest on the person's face;
extract an rPPG signal for each region of interest based on the video;
extract an inter-beat interval (IBI) signal based on a weighted combination of the rPPG signals corresponding to the regions of interest; and
extract the rPPG-based respiratory signal based on the IBI signal.

19. The non-transitory machine-readable medium of claim 15, wherein:
the machine learning model is a binary classifier model; and
the non-transitory machine-readable medium further contains instructions that when executed cause the at least one processor to:
access a training dataset comprising multiple training samples, each training sample including a motion-based respiratory signal, an rPPG-based respiratory signal, and a label indicating whether a motion-based RR or an rPPG-based RR is closer to a ground truth RR for that training sample; and
for each training sample:
extract features of the motion-based respiratory signal and the rPPG-based respiratory signal;
provide the features as input to the machine learning model which predicts whether the motion-based RR or the rPPG-based RR is more likely to be closer to the ground truth RR; and
update parameters of the machine learning model based on a comparison of the label and the prediction.

20. The non-transitory machine-readable medium of claim 19, wherein the features include one or more of: a signal noise ratio, a number of peaks, and a skewness.
